(19) United States
(12) Patent Application Publication
(10) Pub. No.: US 2026/0047863 A1
(43) Pub. Date: Feb. 19, 2026

(54) ROTATIONAL ATHERECTOMY DEVICES AND METHODS

(71) Applicant: Cardio Flow, Inc., St. Paul, MN (US)

(72) Inventors: Jihad A. Mustapha, Ada, MI (US); Gary M. Petrucci, Long Lake, MN (US); Ryan D. Welty, Blaine, MN (US)

(21) Appl. No.: 19/296,149

(22) Filed: Aug. 11, 2025

Related U.S. Application Data
(60) Provisional application No. 63/683,409, filed on Aug. 15, 2024.

(51) Int. Cl. A61B 17/3207

(52) U.S. Cl. CPC A61B 17/320758; A61B 2017/320733

(57) ABSTRACT

Some embodiments of a rotational atherectomy device can remove (partially or completely) stenotic lesions in blood vessels by rotating one or more abrasive elements in an orbital path to abrade and breakdown the lesion. In particular implementations, multiple abrasive elements are arranged along a distal portion of a drive shaft with an improved configuration so as to facilitate both efficient navigation into vessels extending from the abdominal aorta such as the common iliac artery, the external iliac artery, the internal iliac artery, the profunda artery, the gluteal artery, and the pudental artery and effective orbital paths for abrading stenotic material in such vessels and junctions between such vessels.

### CROSS-REFERENCE TO RELATED APPLICATIONS

This application claims benefit of priority to U.S. Provisional Application No. 63/683,409, filed on Aug. 15, 2024, the contents of this aforementioned application are hereby incorporated by reference in its entirety.

### TECHNICAL FIELD

This document relates to rotational atherectomy devices and systems for removing or reducing stenotic lesions in blood vessels, for example, by urging one or more abrasive elements in an orbital motion within the vessel to remove (partially or completely) the stenotic lesion material.

### BACKGROUND

Atherosclerosis, the clogging of arteries with plaque, is often a result of coronary heart disease or vascular problems in other regions of the body. Plaque can be formed from fat, cholesterol, calcium, and other substances found in the blood. Over time, the plaque hardens and narrows the arteries. This limits the flow of oxygen-rich blood to organs and other parts of the body.

Blood flow through the central and peripheral arteries (e.g., carotid, iliac, femoral, renal, etc.) can be similarly affected by the development of atherosclerotic blockages. For example, peripheral artery disease (PAD) can be serious because without adequate blood flow, the kidneys, legs, arms, and feet may suffer irreversible damage. Left untreated, the tissue may die or harbor infection. In another example, coronary artery disease (CAD) arises from the buildup of atherosclerotic material in one or more coronary arteries and may result in a deprivation of blood, oxygen, and nutrients to the heart muscle.

Rotational atherectomy can be used to treat such blockages in some types of blood vessels. In some versions of rotational atherectomy, a drive shaft carrying an abrasive burr or other abrasive surface (e.g., having diamond grit or diamond particles) rotates at a high speed within the vessel, and the clinician operator slowly advances the atherectomy device distally so that the abrasive burr scrapes against the occluding lesion and grinds it into very small particles, reducing the occlusion and improving blood flow through the vessel. For some categories of patients, such a rotational atherectomy process can be obstructed across junctions between larger arteries and smaller arteries or otherwise obstructed in arteries having a tortuous path such as those extending from the abdominal aorta (e.g., the common iliac artery, the external iliac artery, the internal iliac artery, the profunda artery, the gluteal artery, and the pudental artery).

### SUMMARY

Some embodiments of rotational atherectomy systems described herein can remove (partially or completely) stenotic lesions in blood vessels by rotating one or more abrasive elements in an orbital path to abrade and breakdown the lesion, preferably in a manner that significantly increases vessel compliance around the treatment areas to thereby restore pulsatile blood flow and blood pressure to downstream vessels. In some example described below, the system can safely remove calcified lesions from an interior wall of targeted blood vessels (e.g., such as the common iliac artery, the external iliac artery, the internal iliac artery, the profunda artery, the gluteal artery, and the pudental artery) in a manner that improves vessel compliance to restore more natural flexibility/elasticity of the artery wall that is responsive to the pulsatile pressure from the heart. Optionally, in doing so, some embodiments of the rotational atherectomy systems described herein can achieve a clinically effective method of treating erectile dysfunction, iliac artery disease, claudication (including gluteal claudication), lack of blood flow to tissues of a hip joint, hip pain, thigh pain, or low back pain. In particular implementations, multiple abrasive elements are arranged along a distal portion of a flexible coil drive shaft so as to facilitate both efficient navigation into targeted blood vessels (e.g., those extending from the abdominal aorta such as the common iliac artery, the external iliac artery, the internal iliac artery, the profunda artery, the gluteal artery, and the pudental artery) and effective orbital paths for abrading stenotic material in such vessels. Additionally, some versions of the system described herein can be effective for the removal or reduction of stenotic lesions across junctions between larger vessels and smaller vessels, thereby facilitating a single procedure that advantageously removes of stenotic lesions in progressively smaller vessels. The improved configuration thereby provides the user with options for efficiently treating a variety of arterial sites during a single procedure.

Some embodiments described herein include a rotational atherectomy system. The rotational atherectomy system also includes a rotational atherectomy device configured to remove stenotic lesion material from an iliac artery in a manner that increases vessel compliance, may include: a torque-transmitting coil of one or more filars that are helically wound around in a filar wind direction from a distal end to a proximal end to define a coil diameter and a drive shaft axis; and one or more abrasive burrs fixedly mounted to a distal end portion of the torque-transmitting coil; and a rotational atherectomy handle assembly coupled to a proximal end of the torque-transmitting coil and housing an electric motor configured to, responsive to user input at an actuator of the rotational atherectomy handle assembly, drive rotation of the one or more abrasive burrs about the drive shaft axis in a rotational direction.

Some embodiments described herein include an erectile dysfunction treatment system. The erectile dysfunction treatment system also includes a rotational atherectomy device configured to remove stenotic lesion material from one or more arteries selected from an iliac artery and a pudental artery in a manner that increases vessel compliance, may include: a torque-transmitting coil of one or more filars that are helically wound around in a filar wind direction from a distal end to a proximal end to define a coil diameter and a drive shaft axis, and one or more abrasive burrs fixedly mounted to a distal end portion of the torque-transmitting coil and sized to advance into said one or more arteries selected from an iliac artery and a pudental artery.

Some embodiments described herein include an iliac artery disease treatment system. The iliac artery disease treatment system also includes a rotational atherectomy device configured to remove stenotic lesion material from one or more arteries selected from a common iliac artery, an internal iliac artery, and an external iliac artery in a manner that increases vessel compliance, may include: a torque-transmitting coil of one or more filars that are helically wound around in a filar wind direction from a distal end to a proximal end to define a coil diameter and a drive shaft axis, and one or more abrasive burrs fixedly mounted to a distal end portion of the torque-transmitting coil and sized to advance into said one or more arteries selected from an iliac artery and a pudental artery.

Some embodiments described herein include a claudication treatment system. The claudication treatment system also includes a rotational atherectomy device configured to remove stenotic lesion material from one or more arteries selected from a common iliac artery, an external iliac artery, an internal iliac artery, a profunda artery, a gluteal artery, and a pudental artery in a manner that increases vessel compliance, may include: a torque-transmitting coil of one or more filars that are helically wound around in a filar wind direction from a distal end to a proximal end to define a coil diameter and a drive shaft axis, and one or more abrasive burrs fixedly mounted to a distal end portion of the torque-transmitting coil and sized to advance into said one or more arteries selected from an iliac artery and a pudental artery.

Some embodiments described herein include a gluteal claudication treatment system. The gluteal claudication treatment system also includes a rotational atherectomy device configured to remove stenotic lesion material from one or more arteries selected from an iliac artery and a gluteal artery in a manner that increases vessel compliance, may include: a torque-transmitting coil of one or more filars that are helically wound around in a filar wind direction from a distal end to a proximal end to define a coil diameter and a drive shaft axis, and one or more abrasive burrs fixedly mounted to a distal end portion of the torque-transmitting coil and sized to advance into said one or more arteries selected from an iliac artery and a pudental artery.

Some embodiments described herein include a rotational atherectomy method for removing stenotic lesion material from a common iliac artery of a patient. The rotational atherectomy method also includes advancing a torque-transmitting coil of a rotational atherectomy device over a guidewire and into a common iliac artery so that at least one abrasive burr mounted to a distal end portion of the torque-transmitting coil is proximate to a stenotic lesion within the common iliac artery. The method also includes rotating the torque-transmitting coil of the rotation atherectomy device so that the at least one abrasive burr mounted to the torque-transmitting coil abrades the stenotic lesion within the common iliac artery.

Such a method can include one or more of the following optional features. The method may include: removing the torque transmitting coil from the common iliac artery while maintaining the guidewire in the common iliac artery. The method may include: advancing a balloon instrument over the guidewire and into the common iliac artery; and expanding the balloon within the common iliac artery subsequent to the rotating of the torque transmitting coil of the rotation atherectomy device. The method may include: advancing the guidewire into an internal iliac artery; and rotating the torque-transmitting coil of the rotation atherectomy device so that the at least one abrasive burr mounted to the torque-transmitting coil abrades a stenotic lesion within the internal iliac artery. The method may include: advancing the guidewire into a gluteal artery; and rotating the torque-transmitting coil of the rotation atherectomy device so that the at least one abrasive burr mounted to the torque-transmitting coil abrades a stenotic lesion within the gluteal artery. The method may include: advancing the guidewire into a pudental artery; and rotating the torque-transmitting coil of the rotation atherectomy device so that the at least one abrasive burr mounted to the torque-transmitting coil abrades a stenotic lesion within the pudental artery. The method may include: advancing the guidewire into a common femoral artery; and rotating the torque-transmitting coil of the rotation atherectomy device so that the at least one abrasive burr mounted to the torque-transmitting coil abrades a stenotic lesion within the common femoral artery. The method may include: advancing the guidewire into a profunda artery; and rotating the torque-transmitting coil of the rotation atherectomy device so that the at least one abrasive burr mounted to the torque-transmitting coil abrades a stenotic lesion within the profunda artery.

Some embodiments described herein include a method of treating erectile dysfunction using rotational atherectomy. The method also includes advancing a torque-transmitting coil of a rotational atherectomy device over a guidewire and into a common iliac artery so that at least one abrasive burr mounted to a distal end portion of the torque-transmitting coil is proximate to a stenotic lesion within the common iliac artery. The method also includes rotating the torque-transmitting coil of the rotation atherectomy device so that the at least one abrasive burr mounted to the torque-transmitting coil abrades the stenotic lesion within the common iliac artery.

Some embodiments described herein include a method of treating erectile dysfunction using rotational atherectomy by performing rotational atherectomy in one or more vessels extending from the abdominal aorta including at least one of a common iliac artery.

Some embodiments described herein include a method of treating hip pain using rotational atherectomy by performing rotational atherectomy in one or more vessels extending from the abdominal aorta including at least one of a common iliac artery.

Some embodiments described herein include a method of treating low back pain using rotational atherectomy by performing rotational atherectomy in one or more vessels extending from the abdominal aorta including at least one of a common iliac artery.

Some embodiments described herein include a method of increasing pulsatile blood flow to tissue of a hip joint by performing rotational atherectomy in one or more vessels extending from the abdominal aorta including at least one of a common iliac artery.

Some of the embodiments described herein may provide one or more of the following advantages. First, some embodiments of the rotational atherectomy system can be configured to provide a rotational atherectomy treatment in arteries extending from the abdominal aorta (such as the common iliac artery, the external iliac artery, the internal iliac artery, the profunda artery, the gluteal artery, and the pudental artery) in a manner that eliminates calcified lesions from the interior vessel wall so as to restore vessel compliance and downstream pulsatile blood pressure. In some examples described below, an improved method of increasing vessel compliance and restoring (increasing) natural downstream blood pressure can be effectively achieved using a rotational atherectomy device having a selected the orientation, relative spacing, and relative size of the abrasive elements along the distal end portion of the drive shaft (along with other features of the drive shaft).

Second, some embodiments of the rotational atherectomy devices and systems provided herein can advantageously advance through target arteries extending from the abdominal aorta, including arteries of some patients in which the access path follows a tortuous route (e.g., an access path extending through the common iliac artery and into one or more of the external iliac artery, the internal iliac artery, the profunda artery, the gluteal artery, and the pudental artery). The rotational atherectomy devices and systems can treat, in a single procedure, a combination of the target arteries utilizing a sufficient balance of factors (e.g., length, maximum lateral radius from a central axis, flexibility, torque-transmission capabilities, etc.) to advance into the target arteries to remove (fully or partially) stenotic material from a targeted artery. Treatment of the target arteries and the removal (fully or partially) of stenotic material from the targeted arteries facilitates an increase in the compliance of the treated vessel along with vessels upstream of the treated vessel. Increased compliance of the vessels (e.g., including the removal of calcified plaque to restore vessel compliance of the artery wall and thus more natural flexibility/elasticity that is responsive to the pulsatile pressure from the heart) facilitates an increase of blood flow throughout the treated artery and downstream of the treated area, which can achieve a meaningful treatment for ailments suffered in organs, muscles, and other tissues supplied by the treated vessels in the treatment path. For example, some embodiments of the rotational atherectomy devices and systems provided herein can advantageously facilitate the treatment of erectile dysfunction, low back pain, provide an alternative to a hip replacement surgery, iliac artery disease, hip pain, thigh pain, claudication, among others.

Third, some embodiments of the rotational atherectomy devices and systems provided herein can advantageously utilize the same guidewire to guide both a rotational atherectomy device and an angioplasty balloon device to a targeted vessel. For example, the single guidewire instrument can advance through target arteries extending from the abdominal aorta, such as the common iliac artery, the external iliac artery, the internal iliac artery, the profunda artery, the gluteal artery, and the pudental artery to treat the various target arteries. The guidewire can remain in position in the targeted artery while a drive shaft of the rotational atherectomy device is advanced into the targeted artery atherectomy treatment at the treatment site, and furthermore can remain in the targeted artery while an angioplasty balloon device is subsequently advanced into the targeted artery for angioplasty treatment at the same treatment site. Accordingly, the system can be used to improve procedure efficiency where the atherectomy device and a balloon instrument (e.g., an angioplasty device) can be exchanged over the same guidewire. The exchange of devices facilitates an efficient and customizable treatment process that can utilize both rotational atherectomy and subsequent balloon angioplasty.

Fourth, some embodiments of the rotational atherectomy devices and systems provided herein can advantageously facilitate the treatment of various target areas, including junctions between target arteries including an aorto-iliac junction, a common iliac-internal iliac junction a common femoral-profunda junction, among others. Removal (fully or partially) of stenotic lesions at the various arterial junctions can enhance downstream impact of rotational atherectomy by increasing blood flow to the various vessels downstream from the junction.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 a perspective view of an example rotational atherectomy system, in accordance with some embodiments.

FIG. 2 is a longitudinal side view of a rotational atherectomy device of the system of FIG. 1.

FIG. 3 is a distal end view of the rotational atherectomy device of FIG. 2.

FIG. 4 is a perspective view of the distal portion of the example rotational atherectomy system of FIG. 1 in a blood vessel.

FIG. 5 is a transverse cross-sectional view of a distal tip of the rotational atherectomy device of FIG. 2.

FIG. 6 is a longitudinal side view of an example rotational atherectomy system, in accordance with some embodiments.

FIG. 7 is a longitudinal side view of an example rotational atherectomy system, in accordance with some embodiments.

FIG. 8 is a perspective view of the distal portion of the example rotational atherectomy system of FIG. 1 in a common iliac artery, in accordance with some embodiments.

FIG. 9 is a detailed perspective view of the distal portion of the example rotational atherectomy system of FIG. 8, in accordance with some embodiments.

FIG. 10 is a detailed perspective view of a guidewire positioned in the iliac artery of FIG. 9, in accordance with some embodiments.

FIG. 11 is a detailed perspective view of an example balloon device extending over the guidewire of FIG. 10, in accordance with some embodiments.

FIG. 12 is a perspective view of a distal portion of an example rotational atherectomy system in an internal iliac artery and/or a gluteal artery, in accordance with some embodiments.

FIG. 13 is a perspective view of a distal portion of an example rotational atherectomy system in a pudental artery, in accordance with some embodiments.

FIG. 14 is a perspective view of a distal portion of an example rotational atherectomy system in a profunda artery, in accordance with some embodiments.

FIG. 15 is a perspective view of the distal portion of the example rotational atherectomy system of FIG. 1 in a common iliac artery, in accordance with some embodiments.

Like reference symbols in the various drawings indicate like elements.

### DETAILED DESCRIPTION OF ILLUSTRATIVE EMBODIMENTS

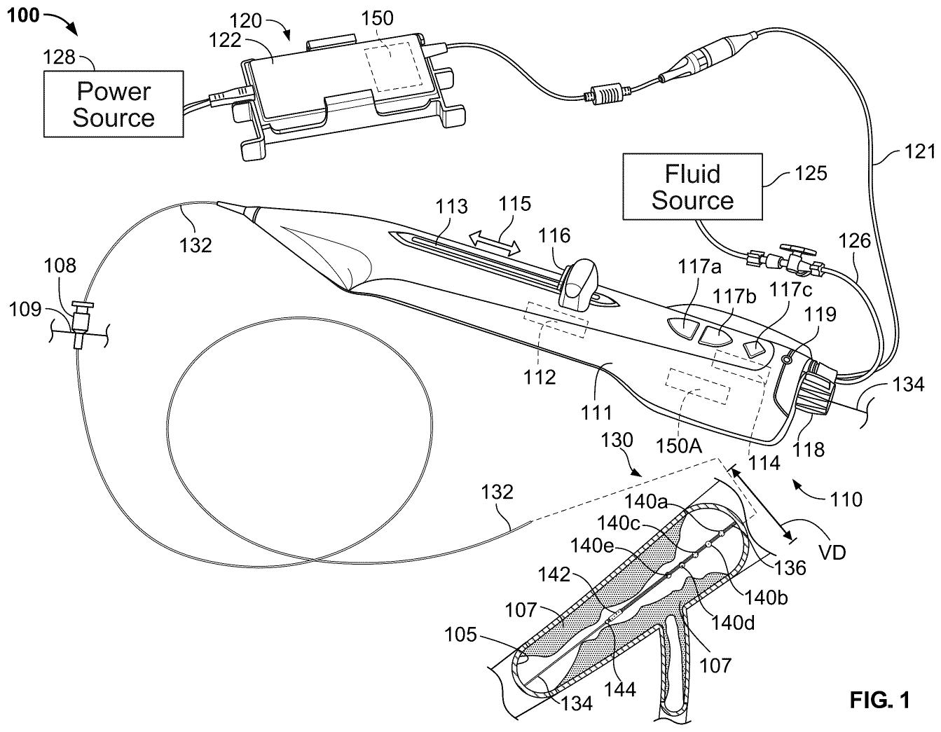

Referring to FIG. 1, in some embodiments a rotational atherectomy system 100 for removing (partially or completely) a stenotic lesion 107 from a targeted blood vessel 105 can include an actuator handle assembly 110 that controls movement of an elongate flexible drive shaft assembly 130. The drive shaft assembly 130 includes a flexible drive shaft 136, and a distal end portion of the driveshaft 136 includes one or more abrasive elements 140a-e configured to abrade the stenotic lesion 107 in the targeted vessel 105.

In the depicted embodiment, the targeted vessel 105 is a common iliac artery, and the system 100 is configured to safely remove the stenotic lesion 107 from the targeted vessel 105 (and, optionally, from branch vessel such as the external iliac artery, the internal iliac artery, the profunda artery, the gluteal artery, and the pudental artery) in a manner that improves vessel compliance to restore or otherwise increase the elasticity response of the artery wall. In doing so, some implementations of the rotational atherectomy system 100 can be used in a restorative method of treating erectile dysfunction, iliac artery disease, claudication (including gluteal claudication), lack of blood flow to tissues of a hip joint, hip pain, thigh pain, or low back pain.

As described in more detail below, the abrasive elements 140a-e can have a selected configuration and relative sizing along the distal end portion of the drive shaft 136 so as to improve navigation into targeted blood vessels that suffer from a significant buildup of calcified plaque or other stenotic material impairing vessel compliance of the artery wall (e.g., including vessels extending from the abdominal aorta, such as the common iliac artery, the external iliac artery, the internal iliac artery, the profunda artery, the gluteal artery, and the pudental artery). Furthermore, the selected configuration and relative sizing of the abrasive elements 140a-e can also achieve, when the drive shaft is rotated, an effective orbital path for abrading the stenotic material 107 in those vessels. Thus, in some implementations, the system 100 can be configured to both remove the calcified plaque along the stenotic lesions 107 (e.g., within the common iliac artery, the external iliac artery, the internal iliac artery, the profunda artery, the gluteal artery, and the pudental artery) and increase the compliance of the treated vessel along with vessels upstream of the treated vessel. For example, the system 100 can remove stenotic lesions 107 within the common iliac artery and increase compliance of the common iliac artery and the abdominal aorta. In some embodiments, the removal of the stenotic lesions 107 facilitates increased vessel compliance where the vessel walls are relieved of restriction/reduced flexibility/calcification imposed upon the vessel walls by the stenotic lesions 107.

Still referring to FIG. 1, the system 100 can also include a power adapter 120 and a fluid source 125 (e.g., a saline bag) connectable to the actuator handle assembly 110, and the actuator handle assembly 110 can house therein an electric motor 112 (configured to drive rotation of the driveshaft 136) and fluid pump 114 (configured to urge a fluid such as saline toward the distal end portion of the driveshaft 136. As described in more detail below, a controller 150 for activating the electric motor 112 and the pump 114 (responsive to inputs at the user interface buttons 116 and 117a-c of the handle assembly 110) can be contained inside a housing 122 of the power adapter 120 so that it is reusable with subsequent handle assemblies after the first handle assembly 110 is discarded (a single-use handle assembly). Alternatively, a controller 150A for operating the electric motor 112 and the pump 114 can be contained within the housing in the handle assembly 110 (in proximity to the electric motor 112 and the pump 114), and the entire handle assembly 110 can be discarded after a single use with a patient. In both options, the handle assembly 110 can be operated by a clinician using a simplified, screenless interface to perform and control the rotational atherectomy procedure (e.g., without a graphic display screen along the handle assembly or on a separate unit connected to the handle assembly).

Optionally, the elongate flexible drive shaft assembly 130 includes a sheath 132 that extends over a majority length of the flexible drive shaft 136 such that the abrasive elements 140a-e on the distal end portion of the drive shaft 136 are positioned distally of a distal-most end of the sheath 132. A proximal end of the sheath 132 is fixed to a distal end of the handle assembly 110. The flexible drive shaft 136 is slidably and rotatably disposed within a lumen of the sheath 132. The flexible drive shaft 136 defines a longitudinal lumen in which a guidewire 134 is slidably disposed. The guidewire 134 can extend through the handle assembly 110, the sheath 132, and the drive shaft 136 such that a proximal end of the guidewire 134 protrudes proximally from a rear port of a guidewire brake 118 at a proximal end of the handle assembly 110 while a distal end of the guidewire 134 extends distally of a distal-most end of the drive shaft 136. In this embodiment, the flexible drive shaft 136 includes a torque-transmitting coil of one or more helically wound filars that defines the longitudinal lumen along a central longitudinal axis. The drive shaft 136 is configured to rotate about the longitudinal axis while the sheath 132 remains generally stationary. Hence, during a rotational atherectomy procedure, the sheath 132 and the guidewire 134 are generally stationary while the flexible drive shaft 136 is controllably moved (e.g., rotating about the longitudinal axis and periodically longitudinally translating proximally and/or distally).

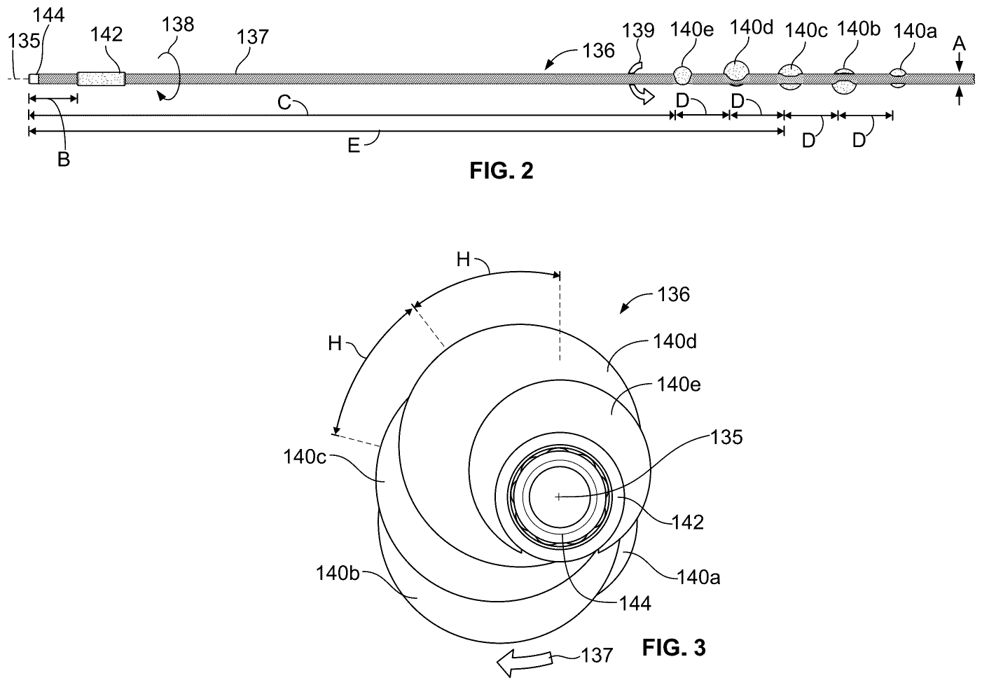

In the depicted embodiment, the exposed distal end portion of the driveshaft 136 includes one or more abrasive elements 140a-e, a (optional) distal stability element 142, and a (optional) concentric tip member 144. In the depicted embodiment, the one or more abrasive elements includes a set of five eccentric abrasive elements 140a-e that are fixedly mounted to an exterior of the torque-transmitting coil of the driveshaft 136 such that a center of massive for each abrasive element 140a-e is offset from a central longitudinal axis of the torque-transmitting coil. In this embodiment, the distal stability element 142 is concentrically-fixed to an exterior of the torque-transmitting coil of the driveshaft 136 between a distal-most one of the eccentric abrasive elements 140a-e and the concentric tip member 144. As such, the center of mass of the distal stability element 142 is aligned with the central axis of the drive shaft 136 while the center of mass of each abrasive element 140a-e is offset from the central axis of the drive shaft 136. The concentric tip member 144 is affixed to, and extends distally from, the terminal distal-most end of the torque-transmitting coil. As described in more detail below, the concentric tip member 144 can have a smoother surface than the abrasive surfaces of the distal stability element 142 and the eccentric abrasive elements 140a-e, and the concentric tip member 144 can be configured to provide initial penetration (and, optionally, dilation) through the stenotic lesion 107 in the targeted vessel 105. Optionally, the one or more abrasive elements 140a-e and drive shaft 136 can have a selected configuration and relative sizing (refer to FIGS. 2-3 in one example) that advantageously provides advancement through a small percutaneous introducer 108 (e.g., sized to slidably receive instruments of 6-French diameter or smaller) at a percutaneous opening 109 along a patient's skin surface, and can further navigate through a stenotic lesion 107 in the targeted vessel 105, such as iliac artery described above, prior to sweeping a larger orbital path (during rotation of the driveshaft 136) for abrading the stenotic material 107.

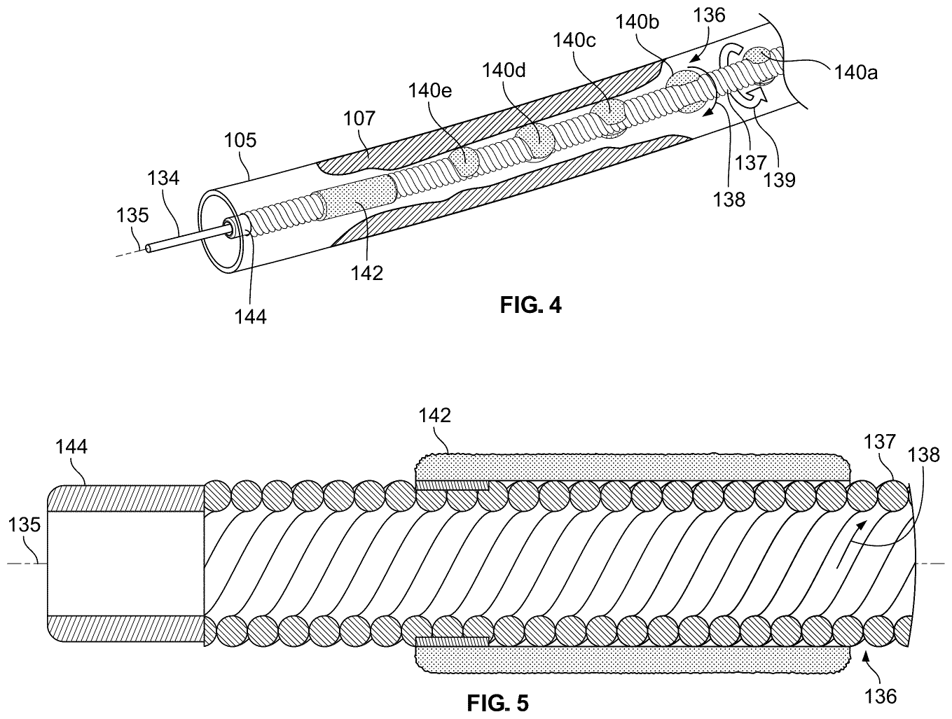

Still referring to FIG. 1, as the drive shaft 136 is rotated about its longitudinal axis, the eccentric abrasive elements 140a-e (and the portion of the drive shaft 136 to which the one or more abrasive elements 140a-e are affixed) will be urged in an orbit path relative to the central axis of the drive shaft 136 (also as described below, for example, in connection with FIGS. 4 and 5). In general, faster speeds (rpm) of rotation of the drive shaft 136 will result in larger diameters of the orbit (within the limits of the vessel diameter). The orbiting one or more abrasive elements 140a-e will contact the stenotic lesion 107 to abrade the lesion to a reduced size with each traversal path through the lesion 107 (i.e., small particles of the lesion will be abraded from the lesion). Depending upon the rotation speed and the surrounding environment within the vessel 105, the rotating distal stability element 142 can remain generally closer to or at the longitudinal axis of the drive shaft 136 during the rotational atherectomy procedure. In some optional embodiments, two or more distal stability elements 142 are included. As described further below, contemporaneous with the rotation of the drive shaft 136, the drive shaft 136 can be translated back and forth (distally and proximally) along the longitudinal axis of the drive shaft 136. Hence, the stenotic lesion 107 can be abraded radially and longitudinally by virtue of the simultaneous translation and orbital rotation of the abrasive elements 140a-e.

Additionally, the torque-transmitting coil of the flexible drive shaft 136 is laterally flexible so that the drive shaft 136 can readily advance through a tortuous arterial path (e.g., within the common iliac artery, the external iliac artery, the internal iliac artery, the profunda artery, the gluteal artery, and the pudental artery), and so that a portion of the drive shaft 136 at, and adjacent to, the one or more abrasive elements 140 can laterally deflect when acted on by the centrifugal forces resulting from the rotation of the one or more eccentric abrasive elements 140. In the depicted embodiment, the drive shaft 136 comprises one or more helically wound wires (or filars) that provides a uniform coil diameter than is less than the diameters of all of the abrasive elements 140a-e and the distal stability element 142. As described in more detail below, this relative sizing is referred to as the burr-to-coil diameter ratio, and the burr-to-coil diameter ratio can be about 1.3-1.7 for all abrasive burrs (elements 140a-e and distal stability element 142) along the torque-transmitting coil of the drive shaft. As such, the torque-transmitting coil of the flexible drive shaft 136 can achieve both sufficient lateral flexibility during navigation through a tortuous path (e.g., including one or more arteries extending from the abdominal aorta, such as the common iliac artery, the external iliac artery, the internal iliac artery, the profunda artery, the gluteal artery, and the pudental artery) and sufficient longitudinal rigidity to be pushed through a stenotic lesion (while transmitting torque to rotate the abrasive elements 140a-e) in the targeted artery. In some embodiments, the one or more helically wound wires (filars) of the torque-transmitting coil of the flexible drive shaft 136 comprise a metallic material such as, but not limited to, stainless steel (e.g., 316, 316L, or 316LVM), nitinol, titanium, titanium alloys (e.g., titanium beta 3), carbon steel, or another suitable metal or metal alloy. Any suitable number of individual filars can be included to construct the drive shaft 136. For example, in some embodiments one, two, three, four, five, six, seven, eight, nine, ten, eleven, twelve, thirteen, fourteen, fifteen, or more than fifteen individual filars can be helically wound among each other to make up the drive shaft 136. As described further below, the direction in which the filars of the drive shaft 136 are wound is a design feature that can be selected to obtain desirable, advantageous operational characteristics. For example, the drive shaft 136 can be formed using one or more filars that are wound about the shaft's central axis in a wind direction that is opposite from the rotational direction of the drive shaft 136 urged by the handle assembly 110, which can provide a number of benefits and improved safety during use of the drive shaft 136 with the guidewire 134 in severely constricted arteries or arteries having a tortuous path such as those extending from the abdominal aorta (e.g., the common iliac artery, the external iliac artery, the internal iliac artery, the profunda artery, the gluteal artery, and the pudental artery).

Still referring to FIG. 1, the torque-transmitting coil of the drive shaft 136 in this embodiment defines a hollow central core (e.g., referred to as a central lumen of the drive shaft 136), which can slidably receive the guidewire 134 therein. In some embodiments, the lumen can be used to aspirate particulate or to convey fluids that are beneficial for the atherectomy procedure. In use, the guidewire 134 is advanced to the targeted vessel 105, and then the drive shaft 136 is advanced over the guidewire 134 in order to reach the targeted vessel 105. The guidewire 134 has a length sufficient to extend through the entire drive shaft 136 and the entire handle assembly 110. As such, a proximal end of the guidewire 134 protrudes proximally from the rear port of the guidewire brake 118 at a proximal end of the handle assembly 110 while a distal end of the guidewire 134 extends distally of a distal-most end of the drive shaft 136.

In the depicted embodiment, the concentric tip member 144 is welded or otherwise fixed to a distal-most end of the torque-transmitting coil of the drive shaft 136 (e.g., axially distal of the coil), and the distal stability element 142 is welded or otherwise fixed to the distal-most end of the torque-transmitting coil of the drive shaft 136 (e.g., radially outward of the coil). As described in more detail below, the smooth initial surface of the concentric tip member 144 followed by the abrasive surface on the distal stability element 142 can help facilitate the initial expansion and abrasion of a pilot path through the stenotic lesion 107 in the targeted vessel 105.

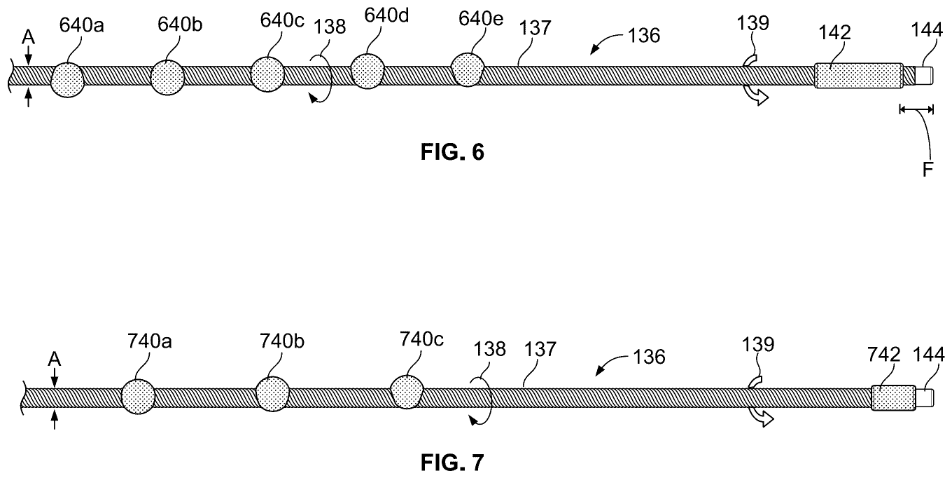

Still referring to FIG. 1, the one or more abrasive elements 140a-e (each of which may also be referred to as an abrasive burr) can comprise a biocompatible material that is coated with an abrasive media such as diamond grit, diamond particles, silicon carbide, and the like. In the depicted embodiment, the abrasive elements 140a-e include a total of five discrete abrasive spheres/cylinders that are spaced apart from each other (and spaced relative to the distal stability element 142) to facilitate both navigation to, and orbital abrading within, a targeted artery, including those where the vessel interior diameter is constricted (severely or fully) and the access path follows a tortuous route. In the depicted embodiment, the abrasive elements 140a and 140e have smaller diameters than abrasive elements 140b, 140c, and 140d. In some embodiments, abrasive elements 140a and 140e can have a same diameter as each other. In some embodiments, abrasive elements 140b, 140c, and 140d can have a same diameter as each other. The diameter of each of abrasive elements 140b, 140c, and 140d can be larger than the diameter of each of abrasive elements 140a and 140e. In some embodiments, all five of abrasive elements 140a-e are spheres are mounted in an eccentric spiral arrangement (described below in connection with FIGS. 2-3). Other embodiments depicted herein (e.g., refer to FIGS. 6-7) can also be used in accordance with the system of 100 of FIG. 1 to facilitate both navigation to, and orbital abrading within, a targeted artery, including those where the vessel interior diameter is constricted (severely or fully) and the access path follows a tortuous route. As with the distal stability element 142, the abrasive burrs 140a-e may be mounted to the exterior of the torque-transmitting coil of the drive shaft 136 using a biocompatible adhesive, high temperature solder, welding, press fitting, and the like. Alternatively, the one or more abrasive elements 140a-e can be integrally formed as a unitary structure with the filars of the drive shaft 136 (e.g., using filars that are wound in a different pattern to create an axially offset structure, or the like).

Still referring to FIG. 1, the rotational atherectomy system 100 also includes the actuator handle assembly 110. The actuator handle assembly 110 includes a housing 111 and an internal carriage assembly (not shown) that translates along an actuator slot 113. For example, a user can grasp the actuator 116 to urge movement along the actuator slot 113, which causes the internal carriage assembly to slidably translate along the longitudinal axis of the handle assembly 110, as indicated by the arrow 115. In some embodiments the carriage assembly can be translated, without limitation, about 8 cm to about 12 cm, or about 6 cm to about 10 cm, or about 4 cm to about 8 cm, or about 6 cm to about 14 cm. As the carriage assembly is translated in relation to the housing 111, the drive shaft 136 translates in relation to the sheath 132 in a corresponding manner. As such, the user can reciprocate the distal end portion of the drive shaft 136 in distal and proximal directions relative to the stenotic lesion 107 within the targeted vessel 105.

The handle assembly 110 has a cable connection 121 with a power adapter 120 (configured to receive electrical power from a power source 128 such as a wall plug) and fluid line connection 126 with a saline source 125. The cable 121 can communicate both power and data (e.g., when the controller 150 is housed within the power adapter housing 122), or alternatively, can communicate electrical power (e.g., when implementing the version with the controller 150A that is housed in the handle housing 111). The cable 121 includes a removable connection jack so that the handle assembly 110 can be readily discarded after a single use and the power adapter 120 can be reused with subsequent handle assemblies. The fluid line connection 126 can include a luer fitting and a flow on-off valve so that a user can removably connect the handle assembly to a pole-mounted saline bag or other fluid source 125 without the need for an external pump mechanism positioned exterior to the handle housing 111.

Still referring to FIG. 1, the actuator 116 of the handle assembly 110 includes a rotational power button that activates the electrical motor 112 (carried by the internal carriage assembly) to drive the rotation of the drive shaft 136. For example, when the rotational power button of the actuator 116 is depressed, power is supplied to the electric motor 112, which is coupled to the drive shaft 136 via a set of gears. It should be understood that the rotational atherectomy system 100 is configured to rotate the drive shaft 136 at a high speed of rotation (e.g., 20,000-160,000 rpm) such that the eccentric one or more abrasive elements 140a-e revolve in an orbital path to thereby contact and remove portions of a target lesion 107 (even those portions of the lesion that are spaced farther from the axis of the drive shaft 136 than the maximum radius of the abrasive elements 140a-e).

To operate the handle assembly 110 during a rotational atherectomy procedure, a clinician can grasp the actuator 116 and depress rotational power button (on the actuator 116) with the same hand. The clinician can move (translate) the actuator 116 along the slot 113 distally and proximally by hand (e.g., back and forth in relation to the housing 111), while maintaining the rotational power button of the actuator 116 in the depressed state. In that manner, a target lesion 107 can be abraded radially and longitudinally by virtue of the resulting orbital rotation and translation of the abrasive elements 140a-e.

To further operate the handle assembly 110 during a rotational atherectomy procedure, a clinician can select a rotational speed using electrical switches 117a and 117b. In some cases, the rotational speed can be selected through a set of predefined speeds (e.g., at least two predefined speed settings, such as “low” and “high”) with electrical switch 117a causing an increase in the speed setting and electrical switch 117b causing a decrease in the speed setting. Optionally, each of the electrical switches 117a-b can also include a light indicator. For example, when the electrical switches 117a-b allow for selection for a “high” and “low” speed, respectively, the electrical switches 117a-b can each have a single light, such that when a speed is selected, the light corresponding to the selected electrical switch 117a or 117b is illuminated to inform a clinician of the selected speed. In some embodiments, the light can shine through electrical switches 117 and 117b. Alternatively, a light can be positioned proximal electrical switch 117a-b. As another example, when the electrical switches 117a-b allow modification of a speed between a range of speeds, the light indicator can be a light bar, such that a number of lights illuminated on the light bar correspond to a selected speed.

Still referring to FIG. 1, handle assembly 110 can include a fluid pump switch 117c, which can activate the internal fluid pump 114 to draw fluid (e.g., saline in this embodiment) from the fluid line 126 and urge the fluid through the sheath 132 toward the distal end portion of the drive shaft 136. As such, the fluid pump switch 117c can be used to both initially prime the sheath 132 (and remove air before insertion into the patient) and then selectively activate additional flush fluid through the sheath 132 and into the vessel 105. In some cases, a first depression of the fluid pump switch 117c will turn the internal pump 114 on, while a second depression will turn the pump 114 off. In some embodiments, the fluid pump switch 117c includes a light indicator, such that when the pump is on, a light is illuminated to inform the clinician that the pump is on.

In the depicted embodiment, the handle assembly 110 also includes a guidewire brake 118 that can be selectively actuated (e.g., pivoted relative to the handle housing 111 in this embodiment) to releasably clamp the guidewire 134 in a stationary position relative to the handle assembly 110 (and, in turn, stationary in relation to rotations of the drive shaft 136 during an atherectomy treatment). While the drive shaft 136 and handle assembly 110 are being advanced over the guidewire 134 to put the one or more abrasive elements 140 into a targeted position within a patient's vessel, the guidewire brake 118 is in a non-activated state (e.g., pivoted counter-clockwise about the central guidewire axis) from a rear perspective) so that the handle assembly 110 is free to slide in relation to the guidewire 134. Then, when the clinician is ready to begin the atherectomy treatment, the guidewire brake 118 can be activated (e.g., pivoted clockwise about the central guidewire axis) to mechanically engaged an exterior of the guidewire 134 and thereby releasably detain/lock the guidewire 134 in relation to the handle assembly 110. That way the guidewire 134 will not rotate while the drive shaft 136 is rotating, and the guidewire 134 will not translate while the actuator 116 is being manually translated in the direction 115.

Still referring to FIG. 1, handle assembly 110 can include a guidewire brake light 119 that positioned along an upper face of the handle housing 111 at a position proximal to the other user interface buttons 117a-c and adjacent to the guidewire brake 118. As such, a user can readily view the guidewire brake light 119 and receive confirmation of whether the guidewire brake 118 is fully activated (to clamp the guidewire 134) before selecting the rotational speed (e.g., buttons 117a-b) and activating rotation (e.g., button on the actuator 116). As such, the screenless user interface of the handle assembly 110 can provide a simplified and fluid hand motion for the user while also communicating effective information to the user. Optionally, the controller 150 (or 150A in other embodiments) can be configured to prevent the electric motor 112 from driving rotation of the drive shaft 136 until: (1) the guidewire brake 118 is activated (e.g., with the guidewire brake light 119 illuminated), (2) the pump 114 is activated to drive the flush fluid (e.g., via actuation of fluid pump switch 117c that then illuminates the button 117c), (3) a rotation speed has been selected via speed selection switches 117a and 117b (e.g., with a speed indicator light thereon being activated), or a combination of all these conditions. As another example, the indicator lights associated with the selection switches 117a and 117b, the fluid pump switch 117c, and the guidewire brake light 119 will alert a clinician that the rotational atherectomy system 100 should not be operated until all three systems (the motor, the pump, the guidewire brake) are activated. For example, each system may have a green light, such that three green lights indicates the clinician can proceed with the atherectomy procedure. Optionally, only the guidewire 118 needs to be actuated to allow rotation of the rotational atherectomy system 100.

Still referring to FIG. 1, the rotational atherectomy system 100 also includes the controller 150, which in this embodiment which includes a processor and computer-readable memory storing control instructions thereon. The controller 150 is configured to receive input from sensors housed within the handle assembly, to receive input from the user interface on the handle assembly 110 (e.g., switches/actuators 116, 117a-c, and 118), and to control the activation of the electric motor 112 and the pump 114 (responsive to inputs at the user interface switches/actuators). In this embodiment, the controller 150 is contained inside the housing 122 of the power adapter 120 so that it is reusable with subsequent handle assemblies after the first handle assembly 110 is discarded (e.g., after use with a first patient). As previously described, the cable 121 can provide data communication between the controller 150 and the components of the user interface (e.g., switches/actuators 116, 117a-c, and 118), the electric motor 112, the pump 114, and the feedback sensors housed within the handle assembly 110. In an alternative embodiment, the controller (including the processor and computer-readable memory storing the control instructions) can be provided in the form of controller 150A configured to be contained within the housing 111 of the handle assembly 110 (in proximity to the electric motor 112 and the pump 114). In both options, the handle assembly 110 can be operated by a clinician using the above-described simplified, screenless interface to perform and control the rotational atherectomy procedure (e.g., without a user interface display screen along the handle assembly or on the units connected to the handle assembly). Preferably, the controller 150 (or controller 150A) is housed in a manner that is sealed from fluids encountered by the handle assembly, such as saline, blood, or others.

Referring now to FIGS. 2-3, some embodiments of the distal end portion of the drive shaft 136 include an improved configuration of the abrasive burrs (and optionally the distal stability element) that provide a relative orientation, relative spacing, and relative sizing along the torque-transmitting coil 137 of the drive shaft 136 so as to achieve an efficient access path to such arteries distal to the common iliac artery, including those where the vessel interior diameter is constricted (severely or fully) and the access path follows a tortuous route. In the depicted embodiment, the abrasive elements 140a-e are eccentrically-fixed to the torque-transmitting coil 137 of the driveshaft 136 while the distal stability element 142 (having a similar abrasive surface) is concentric with the torque-transmitting coil 137 of the drive shaft 136.

The abrasive elements 140a-e are arranged at differing radial angles in relation to the drive shaft 136 as depicted here. In such a case, a path defined by the centers of mass of the abrasive elements 140a-e spirals along the drive shaft 136 around the central longitudinal axis of the drive shaft 136. In some cases (e.g., when the diameters of the abrasive elements 140a-e are equal and the adjacent abrasive elements are all equally spaced), the centers of mass of the abrasive elements 140a-e define a helical path along/around the drive shaft 136. The arrangements of the abrasive elements 140a-e around the drive shaft 136 can facilitate orbital rotation of the abrasive elements 140a-e.

In the depicted embodiment, the two outermost abrasive elements (e.g., abrasive elements 140a, 140e) are smaller in maximum diameter than the three inner abrasive elements (e.g., abrasive elements 140b-d). Optionally, in some embodiments, all of the abrasive elements can be the same size. In particular embodiments, three or more different sizes of abrasive elements are included. Any and all such possible arrangements of sizes of abrasive elements are envisioned and within the scope of this disclosure.

The abrasive elements 140a-e can be made to any suitable size. For clarity, the size of the abrasive elements 140a-e will refer herein to the maximum outer diameter of individual abrasive elements of the abrasive elements 140a-e. In some embodiments, the abrasive elements 140a-e are about 2 mm in size (maximum outer diameter). In some embodiments, the size of the abrasive elements 140a-e is in a range of about 1.5 mm to about 2.5 mm, or about 1.0 mm to about 3.0 mm, or about 0.5 mm to about 4.0 mm, without limitation. Again, in a single embodiment, one or more of the abrasive elements 140a-e can have a different size in comparison to the other abrasive elements 140a-e. In some embodiments, the two outermost abrasive elements are about 1.5 mm in diameter and the inner abrasive elements are about 2.0 mm in diameter.

It should be understood that any of the structural features described in the context of one embodiment of the rotational atherectomy devices provided herein can be combined with any of the structural features described in the context of one or more other embodiments of the rotational atherectomy devices provided herein. For example, the size, spacing, and/or shape features (and any other characteristics) of the one or more abrasive elements 140a-e described in the context of FIGS. 1 and 2 can be incorporated in any desired combination with the spiral arrangement of the one or more abrasive elements 140.

In some embodiments, the drive shaft 136 includes the five abrasive elements 140a-e attached to a distal end portion of the drive shaft 136 and each abrasive element has a center of mass offset from the longitudinal axis of the drive shaft 136. A spiral path defined by connecting the centers of mass of the abrasive elements 140a-e spirals around the longitudinal axis 135 of the drive shaft 136. An overall radial angle of the spiral path is defined by a radial angle between a distal-most abrasive element of the abrasive elements 140a-e and a proximal-most abrasive element of the abrasive elements 140a-e. In some embodiments, the overall radial angle of the spiral path of the abrasive elements 140a-e is always less than 180 degrees along any 10 cm length of the distal end portion of the drive shaft 136. In some embodiments, the overall radial angle of the spiral path of the abrasive elements 140a-e is always less than 170 degrees, or less than 160 degrees, or less than 150 degrees, or less than 140 degrees, or less than 130 degrees, or less than 120 degrees, or less than 110 degrees, or less than 100 degrees, or less than 90 degrees along any 10 cm length of the distal end portion of the drive shaft 136.

As shown in FIGS. 2-3, the torque-transmitting coil 137 as a coil diameter A and all of the abrasive burrs 140a-e have larger diameters than the coil diameter A. For example, the abrasive burrs 140a-e can have a diameter of 1.0 mm to 1.33 mm, or 1.1 mm to 1.3 mm, and 1.25 mm (e.g., nominal diameter of 1.25 mm before application of thin abrasive coating). And, in such examples, the coil diameter A can be 0.7 mm to 0.9 mm, or 0.8 mm. In some implementations, the burr-to-coil diameter ratio can be about 1.3-1.7 for all abrasive elements along the torque-transmitting coil of the drive shaft. In the depicted example, the burr-to-coil diameter ratio can be 1.5-1.6 for each eccentric abrasive element 140a-e mounted to the torque-transmitting coil 137 and 1.3-1.4 for the distal stability element 142 mounted to the torque-transmitting coil 137.

Also shown in FIG. 3, the centers of mass of the abrasive burrs 140a-e are offset in different planes at radial spacing angle H. For example, the radial spacing angle H of 5 degrees to 87.5 degrees, preferably 20 degrees to 60 degrees, and 37.5 degrees in the depicted embodiment here. As such, the combined radial angles of the all abrasive burrs 140a-e is less than 175 degrees (e.g., less than 120 degrees, and preferably less than 90 degrees in the depicted embodiment) along that particular length of the drive shaft carrying the abrasive burrs 140a-e.

Still referring to FIGS. 2-3, the abrasive burrs 140a-e can be mounted to the coil 137 so as to provide a length E between a middle abrasive element 140c and the distal-most tip of the drive shaft 136, which can be effective in treating stenotic lesions 107 in such as those in arteries extending from the abdominal aorta (e.g., the common iliac artery, the external iliac artery, the internal iliac artery, the profunda artery, the gluteal artery, and the pudental artery), especially where the access path follows a tortuous route. For example, the length E can be 3 inches or less from the distal-most tip of the drive shaft 136, preferably 2.5 inches or less from the distal-most tip of the drive shaft 136, and about 2.38 inches in the depicted embodiment. Such an end length E can be relatively compact compared to the overall length of torque-transmitting coil 137. For example, the overall length can be 150 cm to 250 cm, 110 cm to 200 cm, and about 197 cm (77.5 inches). As such, the length E can be less than 1% of the overall length B. In some embodiments, the ratio of the overall length B to the end length E is greater than 50:1, about 90:1 to 140:1, and about 103:1 in the depicted embodiment.

In some embodiments, the abrasive burrs 140a-e include a total of five discrete elements that are spaced apart from each other. In other embodiments, one, two, three, four, or five discrete abrasive elements. The relative spacing of the abrasive burrs 140a-e can advantageously affect the orbital path of the abrasive elements, the flexibility of the intermediate sections of the torque-transmitting coil (with such flexibility being useful during advancement through tortuous arterial paths), or both. For example, the distal-most abrasive burr 140e can be spaced from the distal-most end of the shaft by an extension length C, with the next abrasive burr 140d being spaced therefrom by a burr spacing distance D. The middle abrasive burr 140c being spaced from the abrasive burr 140d and the abrasive burr 140b by the burr spacing distance D. The abrasive burr 140b being spaced from the abrasive burr 140a the burr spacing distance D. In the depicted embodiment, the extension length C is greater than the burr spacing distance D. For example, the length C can be 1.0 to 4.0 inches, 1.5 to 3.5 inches, 1.8 to 3.2 inches, 2.0 to 3.0 inches, 2.1 to 2.5 inches, and about 2.38 inches. The burr spacing distance D can be 0.100 to 0.300 inches, 0.100 to 0.200 inches, 0.130 to 0.190 inches, 0.140 to 0.180 inches, 0.150 to 0.170 inches, and about 0.168 inches.

Still referring to FIGS. 2-3, some embodiments of the abrasive burrs 140a-e, the optional distal stability element 142, and the optional distal tip member 144 can provide a relative orientation, relative spacing, and relative sizing along the torque-transmitting coil 137 so as to advantageously advance through a relatively small percutaneous access point 109 using the introducer sheath 108 (e.g., refer to FIG. 1 above) for subsequent advancement into subsequent arteries to remove (fully or partially) stenotic material from a targeted artery. In some embodiments, a distal end of the distal stability element 142 is spaced apart from a distal-most end of the drive shaft 136 by a stability distance B. The stability distance B can be 0.05 to 0.20 inches, 0.07 to 0.17 inches, 0.10 to 0.20 inches. 0.12 to 0.17 inches, or 0.15 inches.

Referring now to FIG. 4, the filar wind direction 138 (e.g., the winding direction of filars of the torque transmitting coil 137 traversing from the distal end toward the proximal end) and the rotational direction 139 of the torque transmitting coil 137 (e.g., the rotational direction of the drive shaft 136 urged by the handle assembly 110) can be configured to provide a number of performance benefits during the rotational atherectomy in arteries extending from the abdominal aorta (e.g., the common iliac artery, the external iliac artery, the internal iliac artery, the profunda artery, the gluteal artery, and the pudental artery), especially where the access path follows a tortuous route. For example, as shown in FIG. 4 (and also in FIGS. 2-3), the filar wind direction 138 is opposite from the rotational direction 139 of the torque transmitting coil 137, which can provide functional benefits when advancing into tight lesions in arteries or through a tortuous path into arteries extending from the abdominal aorta (e.g., the common iliac artery, the external iliac artery, the internal iliac artery, the profunda artery, the gluteal artery, and the pudental artery). In particular, during rotation of the driveshaft 136 adjacent to such a lesion, one of the abrasive elements 140a-e, the distal stability element 142, or the distal tip 144 may catch on the bodily material or otherwise may be momentarily restrained from rotation (even though the proximal end of the drive shaft 136 is rotated by the handle assembly 110 (FIG. 1). In such circumstances, the filar wind direction 138 is opposite from the rotational direction 139 of the torque-transmitting coil 137 as shown in FIGS. 2-3, so the torque-transmitting coil 137 will not suffer a narrowed coil diameter that causes the torque-transmitting coil 137 to clamp down on the guidewire 134 (e.g., creating reduced flexibility and potentially other concerns in the narrow space with the vessel), but instead the coil diameter will remain the same or be momentarily expanded (safely avoiding a clamping effect on the guidewire 134). Optionally, as illustrated in FIG. 4, the abrasive burrs 140a-e can be positioned relatively closer to the distal tip 144.

Accordingly, the drive shaft 136 of the rotational atherectomy system 100 can have a configuration that provides safe and repeatable navigation into smaller blood vessels extending from the abdominal aorta or proximal to the abdominal aorta and effective orbital paths for abrading stenotic material in such smaller vessels. Such configurations can be particularly useful, for example, when implemented as an aorto-iliac junction navigable device, a common iliac-internal iliac junction navigable device, a common femoral-profunda navigable device, among others.

Referring now to FIG. 5, the drive shaft 136 of the rotational atherectomy device can be equipped with the concentric tip member 144 and the distal stability element 142. The concentric tip member 144 can cover the distal-most end of the torque-transmitting coil 137 while the distal stability element 142 is spaced apart from the distal most end of the torque transmitting coil (e.g., by stability distance B). As such, the stenotic lesion 107 (FIG. 4) is initially engaged with the concentric tip member 144 before engaging an exterior of the torque-transmitting coil 137. In this embodiment, the distal tip member is welded (e.g., a butt weld) or otherwise fixed to a distal-most end of the torque-transmitting coil 137 of the drive shaft 136 so that the entirety of the distal tip member 144 is positioned axially distal of the torque-transmitting coil 137. Also, the distal stability element 142 is a cylindrical structure in the embodiment, which is welded or otherwise fixed to the torque-transmitting coil 137 so that the distal stability element 142 is positioned radially outward of the coil diameter. As such, the distal-most end of the torque-transmitting coil of the drive shaft 136 is concealed the concentric tip member 144.

In this embodiment, both the distal stability element 142 and the concentric tip member 144 comprise metallic cylindrical members that are axially aligned with a central axis of the drive shaft 136, but they are different in size and in abrasiveness. The distal stability element 142 has an inner diameter that surrounds an exterior coil diameter of the drive shaft 136 (and thus the maximum outer diameter of the distal stability element 142 is larger than the coil diameter), and the concentric tip member 144 has an outer diameter that is substantially the same as the coil diameter. Also, the distal stability element 142 has an abrasive outer coating. For example, in some embodiments, a diamond coating (or other suitable type of abrasive coating) is disposed on the outer surface of the distal stability element 142. The concentric tip member 144 in this embodiment has smooth exterior surface that is less abrasive than that of the distal stability element 142. In some cases, the smooth initial surface of the concentric tip member 144 followed by the abrasive surface on the distal stability element 142 can help facilitate the initial expansion and abrasion pilot path through the stenotic lesion 107 in the targeted vessel 105. Both the distal stability element 142 and the concentric tip member 144 may comprise a biocompatible material, such as a higher-density biocompatible material. For example, in some embodiments, each of the distal stability element 142 and the concentric tip member 144 may comprise a metallic material such as stainless steel, tungsten, molybdenum, iridium, cobalt, cadmium, and the like, and alloys thereof. Also, in this embodiment, the distal stability element 142 and the concentric tip member 144 have a fixed outer diameter. That is, the distal stability element 142 and the concentric tip member 144 are not an expandable member in the depicted embodiment.

Referring now to FIG. 6, some embodiments of the distal end portion of the drive shaft 136 include an alternative configuration of the abrasive burrs that also provides a relative orientation, relative spacing, and relative sizing along the torque-transmitting coil 137 of the drive shaft 136 so as to achieve an efficient access path to such target arteries or through a tortuous path into arteries extending from the abdominal aorta (e.g., the common iliac artery, the external iliac artery, the internal iliac artery, the profunda artery, the gluteal artery, and the pudental artery). In the depicted embodiment, the components along the distal end portion of the drive shaft 136 are similar to those described in connection with FIGS. 2-3, except that the distal and proximal abrasive elements 640a and 640e have a same size as the intermediate abrasive elements 140b-d, and the (optional) distal stability element 142 is positioned at a smaller stability distance F than stability distance B. For example, the torque-transmitting coil 137, the filar wind direction 138, the rotation direction 139, and the optional distal tip member 144 have a similar configuration as previously described in connection with FIGS. 2-3.

Here, as shown in FIG. 6, the set of abrasive burrs along the end length of the torque-transmitting coil 137 include distal and proximal abrasive burrs 640a and 640e, which have a size that is the same as the intermediate abrasive burrs 640b-d. For example, the abrasive burrs 640a-e can have a diameter of 1.2 mm to 1.33 mm, and particularly 1.25 mm in the depicted embodiment here (e.g., nominal diameter of 1.25 mm before application of thin abrasive coating). As previously described, in such examples, the coil diameter A can be 0.7 mm to 0.9 mm, and 0.8 mm. Accordingly, the burr-to-coil diameter ratio can be 1.5-1.7 for the abrasive burrs 640a-e mounted to the torque-transmitting coil 137. The centers of mass of the abrasive burrs 640a-e are offset in different planes at radial spacing angle H (in a similar manner as described above with respect to FIGS. 2-3). As previously described in connection with FIGS. 2-3, the radial spacing angle H of 5 degrees to 87.5 degrees, preferably 20 degrees to 60 degrees, and 37.5 degrees in the depicted embodiment here. As such, the combined radial angles of all abrasive burrs 640a-e is less than 175 degrees (e.g., less than 120 degrees, and preferably less than 90 degrees in the depicted embodiment) along the end length of the drive shaft.

Referring now to FIG. 7, some embodiments of the distal end portion of the drive shaft 136 include an alternative configuration of the abrasive burrs that also provides a relative orientation, relative spacing, and relative sizing along the torque-transmitting coil 137 of the drive shaft 136 so as to achieve an efficient access path to such target arteries or through a tortuous path into arteries extending from the abdominal aorta (e.g., the common iliac artery, the external iliac artery, the internal iliac artery, the profunda artery, the gluteal artery, and the pudental artery). In the depicted embodiment, the components along the distal end portion of the drive shaft 136 are similar to those described in connection with FIGS. 2-3 and 6, except that the abrasive elements 740a-c include three abrasive elements that have a same size, and the (optional) distal stability element 742 has a smaller size (e.g., a smaller length along the drive shaft 136) and is positioned adjacent to the optional distal tip member 144 rather than being spaced apart from the distal tip member.

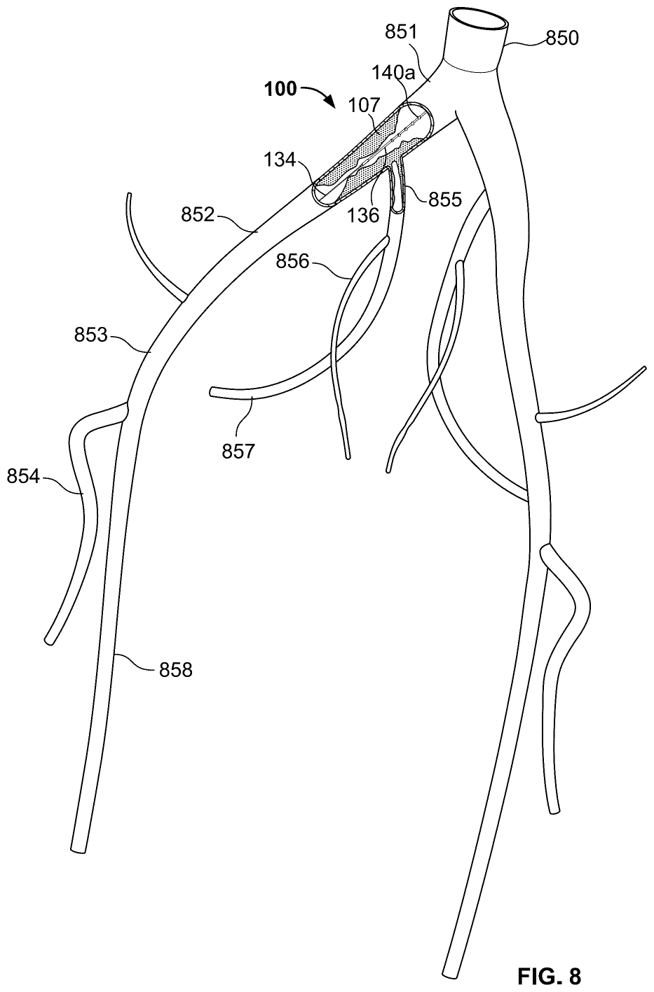

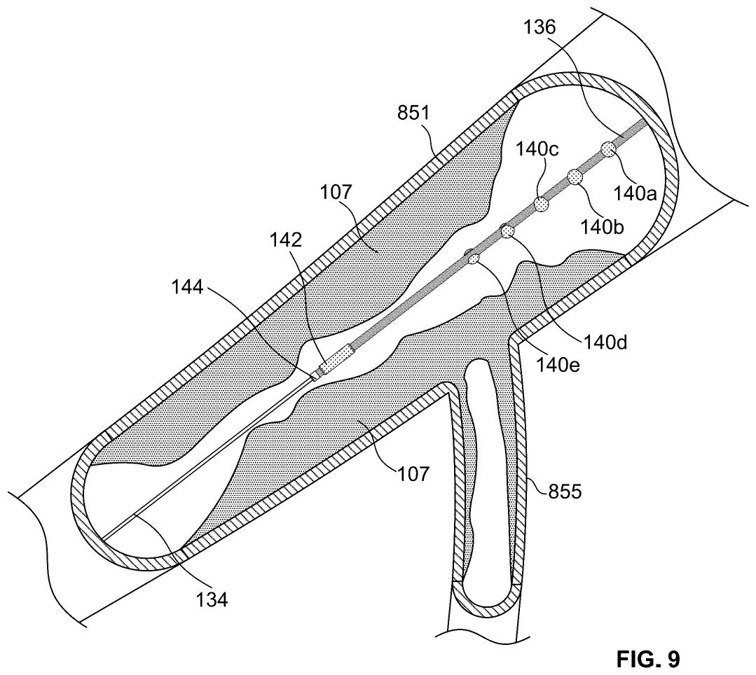

Referring now to FIGS. 8-9, some embodiments of the rotational atherectomy system 100 can remove (partially or completely) one or more stenotic lesions in a targeted artery. For example, in the depicted implementation, the drive shaft 136 of the rotational atherectomy system 100 is navigated through the abdominal aorta 850 artery into the common iliac artery 851. As previously described, the components along the distal end portion of the drive shaft 136 can have an orientation, relative spacing, and relative size so as to achieve an efficient access path to such arteries. In use, the system 100 includes the guidewire 134, which is advanced into the target artery (e.g., to, near, or across the junction between the common iliac artery 851 and the internal iliac artery 855). In the example shown in FIGS. 8-9, the guidewire 134 is navigated through the abdominal aorta 850 into the common iliac artery 851. Such a guidewire position can be useful for rotational atherectomy that targets one or more lesions in the common iliac artery 851 and other target arteries. It should be understood from the description here that the targeted pedal artery may additionally or alternatively include the common iliac artery 851, the external iliac artery 852, the internal iliac artery 855, the profunda artery 854, the gluteal artery 857, the pudental artery 856, and the superficial femoral artery 858. After advancement of the guidewire 134, the drive shaft 136 and sheath 132 (FIG. 1) are advanced over the proximal end of the guidewire 134 such that the proximal end of the guidewire 134 passes through the entirety of the drive shaft 136 and the handle assembly 110 (FIG. 1) to protrude rearwardly from the proximal end of the handle assembly 110. Optionally, the drive shaft 136 and sheath 132 are advanced through a relatively small introducer sheath in a patient's leg.

Still referring to FIGS. 8-9, as the distal end portion of the drive shaft 136 is navigated toward the targeted common iliac artery 851 (preferably, under medical imaging), the concentric tip member 144 (followed by the abrasive surface on the distal stability element 142) can be used to initially form a pilot path through the stenotic lesion 107 in the common iliac artery 851. From there, the user can select a rotational speed setting (e.g., using user interface buttons 117a-b on the handle assembly) so that the abrasive elements 140a-e can abrade the stenotic material and achieve an orbital path during rotation (e.g., an orbital path that is greater in size than a maximum stationary diameter of the abrasive elements 140a-e. The handle assembly 110 (FIG. 1) can be used, via the actuator 116 to translate the abrasive elements 140a-e in reciprocating movement (distally and proximally) for multiple passes through the stenotic lesion in the pedal artery during high-speed rotation of the abrasive elements 140a-e in the orbital path.

Accordingly, the drive shaft 136 of the rotational atherectomy system 100 can have a configuration that provides safe and repeatable navigation into blood vessels along with facilitating effective orbital paths for abrading stenotic material in such vessels. Such configurations can be particularly useful, for example as shown in FIGS. 2-5, and 8-9, when the rotational atherectomy system 100 implemented as a common iliac-internal iliac junction navigable system. Additionally, the implementation of the drive shaft depicted in FIGS. 2-5, and 8-9 can be effective for the removal or reduction of stenotic lesions in larger vessels too (e.g., those in the leg, in the abdominal aorta, and above), thereby providing the user with options for efficiently treating a variety of arterial sites during a single procedure using a single driveshaft 136.

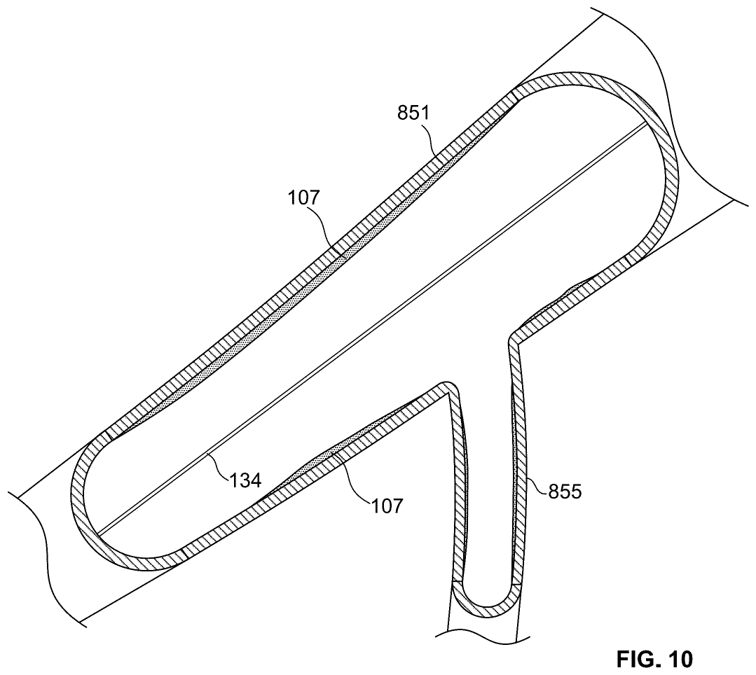

Referring to FIG. 10, the guidewire 134 can remain positioned in the common iliac artery 851 following treatment of the stenotic lesion 107 by the rotational atherectomy system 100. For example, after the rotational atherectomy system 100 is utilized as described above to remove the stenotic lesion 107 the common iliac artery 851 (and optionally additionally in the internal iliac artery 855), the drive shaft 136 can be removed (e.g., over the guidewire 134), while the guidewire 134 remains in place. FIG. 10 shows an example result of the rotational atherectomy system's treatment of the stenotic lesions 107 in the common iliac artery 851 and the internal iliac artery 855, particularly stenotic lesions that extend across or through a junction between the common iliac artery 851 and the internal iliac artery 855. In some embodiments, the stenotic lesions 107 in the common iliac artery 851 and the internal iliac artery 855 are reduced in size (e.g., comparison of FIGS. 9 and 10) as a result of the rotational atherectomy treatment in the area.

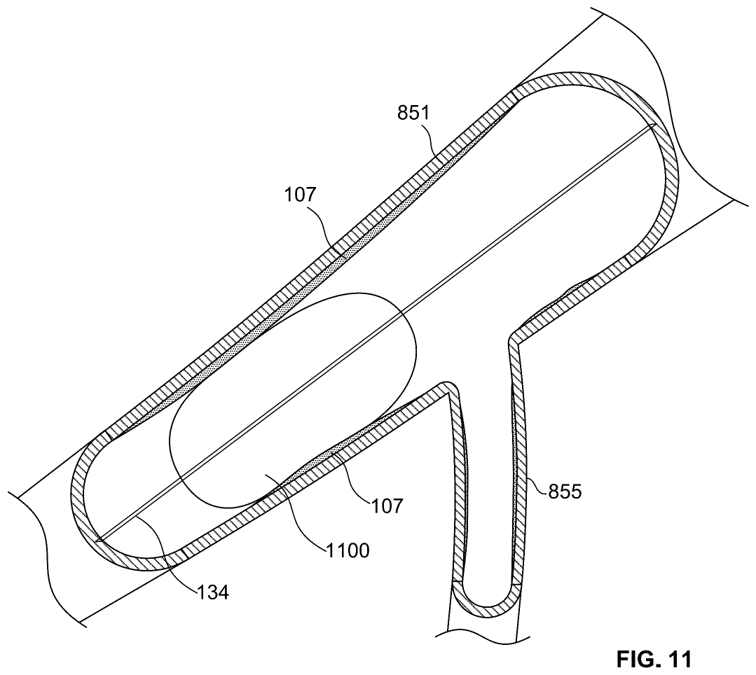

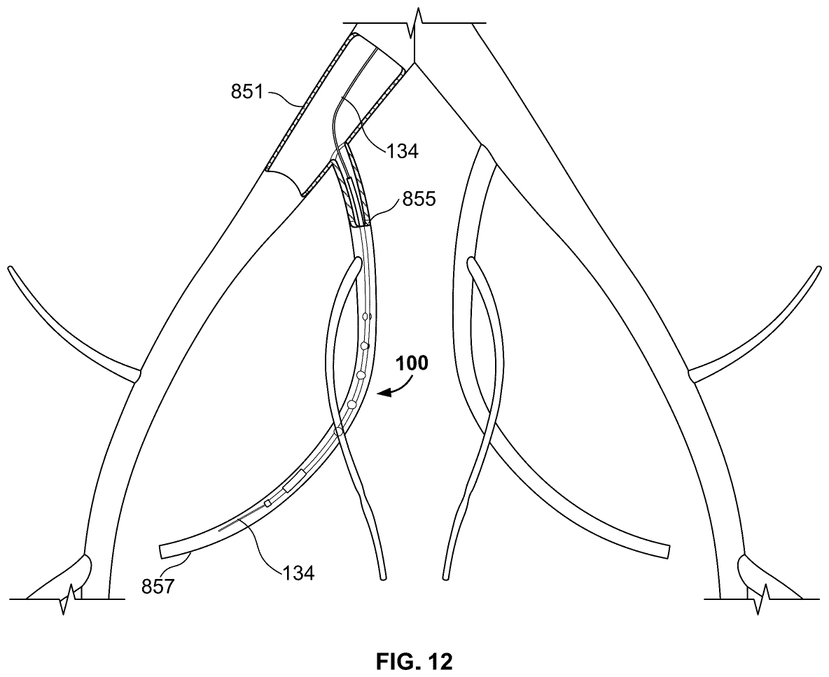

Referring to FIG. 11, the guidewire 134 can remain in position in the common iliac artery 851, and a balloon instrument 1100 can be exchanged over the guidewire 134. For example, subsequent to the removal of the drive shaft 136 from the treatment area, the balloon instrument 1100 can be inserted into the treatment area. During navigation to the treatment area (e.g., the common iliac artery 851, the internal iliac artery 855, and/or across or through a junction between the common iliac artery 851 and the internal iliac artery 855), the balloon instrument 1100 can be deflated such that the balloon is flush against the instrument while the instrument navigates over the guidewire 134 and to the treatment area. When the balloon instrument 1100 is in the treatment area, the balloon can be inflated and extend circumferentially around the guidewire 134 and within the treatment vessel into contact with the remaining portions (if any) of the stenotic lesions 107. The guidewire 134 can facilitate both a convenient and efficient insertion of the balloon instrument 1100 into the treatment area. In some embodiments, the balloon instrument 1100 can facilitate a finishing effect at the treatment area subsequent to the treatment by the rotational atherectomy system 100. For example, the inflation of the balloon instrument 1100 can contact and press the remaining stenotic lesion material against the arterial walls to ensure that the treatment area along the arterial walls is smooth and consistent along the arterial walls.

In some embodiments, the balloon instrument 1100 is optional. For example, the rotational atherectomy system 100 treats the target area and the balloon instrument 1100 is not inserted for subsequent treatment.

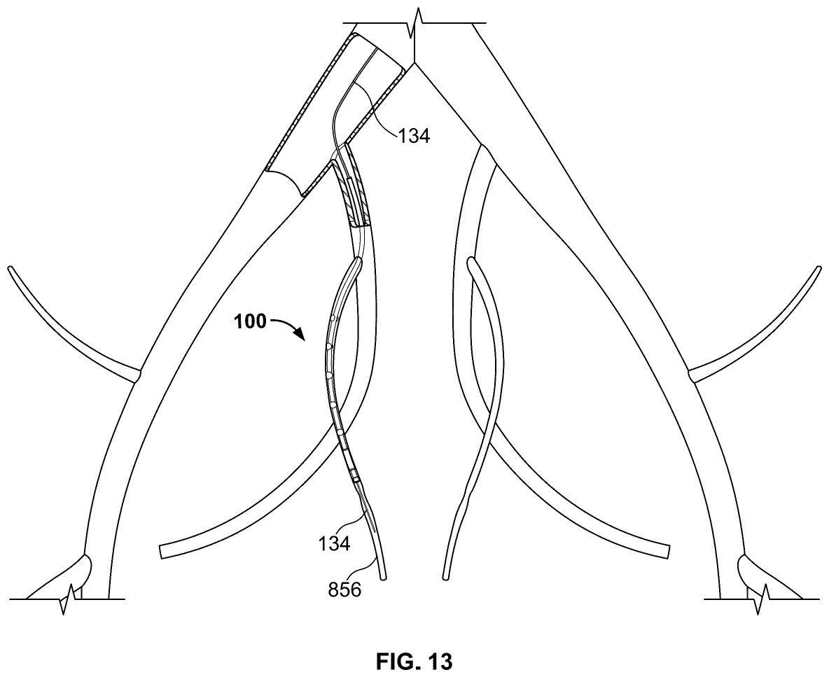

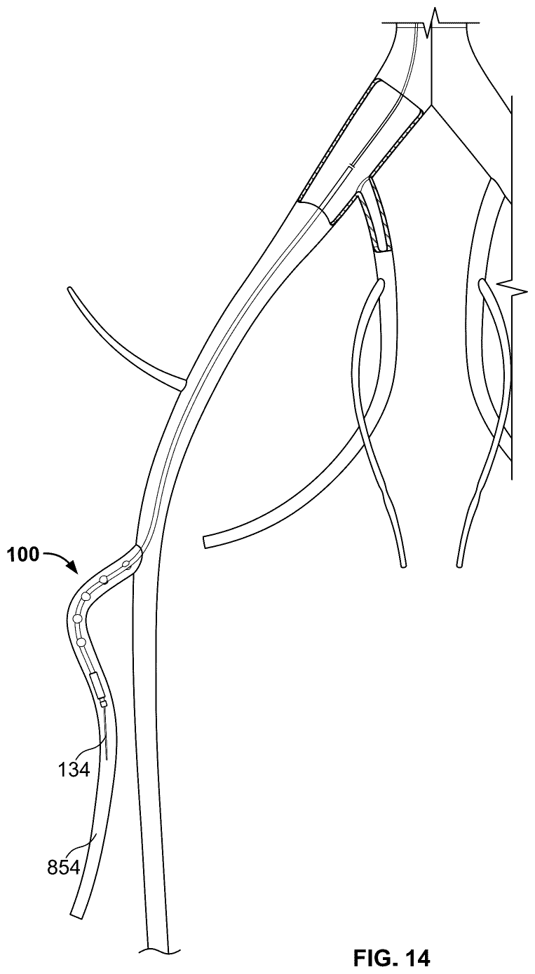

Referring to FIGS. 12-14, additional examples of the rotational atherectomy system 100 are shown inserted into various target vessels for removal of stenotic lesions from such target vessels. For example, the rotational atherectomy system 100 can be subsequently or separately navigated to various target vessels. Alternatively or additionally, different rotational atherectomy systems can be exchanged over the guidewire 134 to treat progressively smaller target areas. For example, the rotational atherectomy system 100 can optionally be removed so that a smaller rotational atherectomy system (e.g., with smaller outer diameter abrasive burrs) can be advanced over the guidewire 134 and to various target vessels. In some embodiments, the rotational atherectomy system 100 (or smaller rotational atherectomy system) can be advanced into the internal iliac artery 855 and/or the gluteal artery 857 (see e.g., FIG. 12). In some embodiments, the rotational atherectomy system 100 (or smaller rotational atherectomy system) can be advanced into the pudental artery 856 (see e.g., FIG. 13). In some embodiments, the rotational atherectomy system 100 (or smaller rotational atherectomy system) can be advanced into the profunda artery 854 (see e.g., FIG. 14). In each of the target arteries, torque-transmitting coil of the rotational atherectomy system 100 (or smaller rotational atherectomy system) can be rotated so that the at least one abrasive burr mounted to the torque-transmitting coil abrades the stenotic lesion within the target vessel.

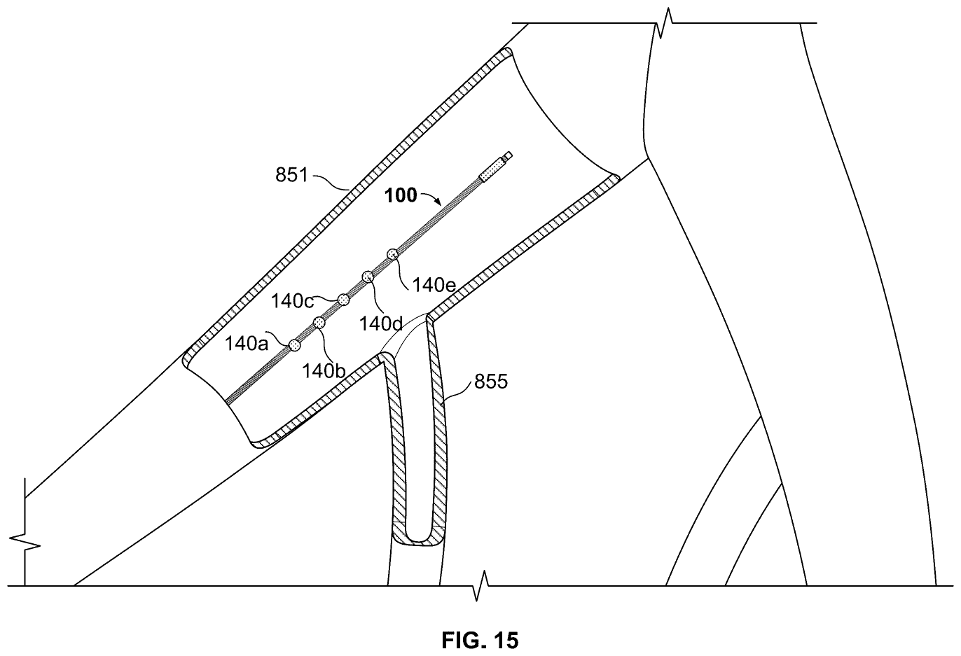

Referring to FIG. 15, some embodiments include the rotational atherectomy system 100 advancing into the common iliac artery 851 in an opposite direction (e.g., with abrasive element 140e positioned anterior to abrasive element 140a).

In some embodiments, the treatment steps described regarding FIGS. 9-10 and optionally FIG. 11, along with the treatment of various other vessels shown in FIGS. 12-15 advantageously facilitate removal of stenotic lesions, plaque, and calcification of target vessels. The treatment steps described herein facilitate the treatment of target arteries and the removal (fully or partially) of stenotic material from the targeted arteries. This removal and treatment facilitates an increase in the compliance of the treated vessel along with vessels upstream of the treated vessel. Increased compliance of the vessels facilitates an increase of blood flow throughout the treated artery and downstream of the treated area. The increased blood flow throughout the treated area and downstream advantageously benefits the organs, muscles, and tissues that are supplied by the vessels in the treatment path. For example, some embodiments of the rotational atherectomy devices and systems provided herein can advantageously facilitate the treatment of erectile dysfunction, low back pain, provide an alternative to a hip replacement surgery, iliac artery disease, hip pain, thigh pain, claudication, among others.

The removal of calcified plaque, stenotic lesions, and other blockages within the target arteries can transform a rigid arterial wall due to the calcification and blockage of the vessel into a pliable and compliant vessel. The compliant vessel can facilitate increased blood flow by being compliant with pulsatile blood flow to facilitate distribution of increased blood flow and blood pressure to downstream vessels. The increase in compliance of the vessels facilitates additional contraction of the vessel and the passing of additional blood flow further downstream. For example, the systems, devices, and methods described herein can be implemented to treat erectile dysfunction. The systems, devices, and methods described herein can facilitate the arterial cleaning of various target vessels (e.g., the common iliac, the internal iliac, and the pudental artery) that provide blood flow to the genital area including the penis. The increased ability for blood to flow to these areas facilitates improved patient outcomes in patients with erectile dysfunction or related symptoms.

Similarly, some embodiments of the rotational atherectomy devices and systems provided herein can advantageously facilitate the treatment of erectile dysfunction, low back pain, provide an alternative to a hip replacement surgery, iliac artery disease, hip pain, thigh pain, claudication, among others.

Some embodiments of the rotational atherectomy devices and systems provided herein can advantageously obviate a stent insertion procedure and provide improved patient outcomes. For example, the rotational atherectomy devices, systems, and methods can remove (partially or fully) stenotic lesions throughout target vessels and across junctions between target vessels. Stent insertion procedures can reduce compliance of the target vessels at least during contraction of the vessel. By obviating the stent insertion and increasing compliance of the target vessels, the rotational atherectomy devices and systems described herein provide improved patient outcomes.

A number of embodiments of the invention have been described. Nevertheless, it will be understood that various modifications may be made without departing from the spirit and scope of the invention. For example, design features of the embodiments described herein can be combined with other design features of other embodiments described herein. Accordingly, other embodiments are within the scope of the following claims.

1-9. (canceled)

10. A rotational atherectomy method for removing stenotic lesion material from a common iliac artery of a patient, comprising:
advancing a torque-transmitting coil of a rotational atherectomy device over a guidewire and into a common iliac artery so that at least one abrasive burr mounted to a distal end portion of the torque-transmitting coil is proximate to a stenotic lesion within the common iliac artery; and
rotating the torque-transmitting coil of the rotation atherectomy device so that the at least one abrasive burr mounted to the torque-transmitting coil abrades the stenotic lesion within the common iliac artery.

11. The method of claim 10, further comprising:
removing the torque-transmitting coil from the common iliac artery while maintaining the guidewire in the common iliac artery.

12. The method of claim 11, further comprising:
advancing a balloon instrument over the guidewire and into the common iliac artery; and
expanding the balloon within the common iliac artery subsequent to the rotating of the torque-transmitting coil of the rotation atherectomy device.

13. The method of claim 12, further comprising:
advancing the guidewire into an internal iliac artery; and
rotating the torque-transmitting coil of the rotation atherectomy device so that the at least one abrasive burr mounted to the torque-transmitting coil abrades a stenotic lesion within the internal iliac artery.

14. The method of claim 13, further comprising:
advancing the guidewire into a gluteal artery; and
rotating the torque-transmitting coil of the rotation atherectomy device so that the at least one abrasive burr mounted to the torque-transmitting coil abrades a stenotic lesion within the gluteal artery.

15. The method of claim 14, further comprising:
advancing the guidewire into a pudental artery; and
rotating the torque-transmitting coil of the rotation atherectomy device so that the at least one abrasive burr mounted to the torque-transmitting coil abrades a stenotic lesion within the pudental artery.

16. The method of claim 12, further comprising:
advancing the guidewire into a common femoral artery; and
rotating the torque-transmitting coil of the rotation atherectomy device so that the at least one abrasive burr mounted to the torque-transmitting coil abrades a stenotic lesion within the common femoral artery.

17. The method of claim 12, further comprising:
advancing the guidewire into a profunda artery; and
rotating the torque-transmitting coil of the rotation atherectomy device so that the at least one abrasive burr mounted to the torque-transmitting coil abrades a stenotic lesion within the profunda artery.

18. A method of treating erectile dysfunction using rotational atherectomy, the method comprising:
advancing a torque-transmitting coil of a rotational atherectomy device over a guidewire and into a common iliac artery so that at least one abrasive burr mounted to a distal end portion of the torque-transmitting coil is proximate to a stenotic lesion within the common iliac artery; and
rotating the torque-transmitting coil of the rotation atherectomy device so that the at least one abrasive burr mounted to the torque-transmitting coil abrades the stenotic lesion within the common iliac artery.

19. The method of claim 18, further comprising:
removing the torque-transmitting coil from the common iliac artery while maintaining the guidewire in the common iliac artery.

20. The method of claim 19, further comprising:
removing the torque-transmitting coil from the common iliac artery while maintaining the guidewire in the common iliac artery.
advancing a balloon instrument over the guidewire and into the common iliac artery; and
expanding the balloon within the common iliac artery subsequent to the rotating of the torque-transmitting coil of the rotation atherectomy device.

21. The method of claim 18, wherein the torque-transmitting coil comprises one or more filars that are helically wound around in a filar wind direction from a distal end to a proximal end to define a coil diameter and a drive shaft axis.

22. The method of claim 21, wherein the at least one abrasive burr mounted to a distal end portion of the torque-transmitting coil comprises a series of abrasive burrs, the series of abrasive burrs including three or more abrasive burrs attached to a distal end portion of the torque-transmitting coil and each having a center of mass offset from the drive shaft axis.

23. The method of claim 22, wherein the three or more abrasive burrs are mounted along the torque-transmitting coil relative to one another so that the centers of mass of the three or more abrasive burrs define a spiral direction that is opposite of the filar wind direction, and the torque-transmitting coil has an outer coil diameter and each abrasive burr in the series of abrasive burrs has a respective burr diameter, wherein a burr-to-coil diameter ratio defined by each respective burr diameter and the outer coil diameter is about 1.3-1.7.

24. The method of claim 10, wherein the torque-transmitting coil comprises one or more filars that are helically wound around in a filar wind direction from a distal end to a proximal end to define a coil diameter and a drive shaft axis.

25. The method of claim 24, wherein the at least one abrasive burr mounted to a distal end portion of the torque-transmitting coil comprises a series of abrasive burrs, the series of abrasive burrs including three or more abrasive burrs attached to a distal end portion of the torque-transmitting coil and each having a center of mass offset from the drive shaft axis.

26. The method of claim 25, wherein the three or more abrasive burrs are mounted along the torque-transmitting coil relative to one another so that the centers of mass of the three or more abrasive burrs define a spiral direction that is opposite of the filar wind direction.

27. The method of claim 26, wherein the torque-transmitting coil has an outer coil diameter and each abrasive burr in the series of abrasive burrs has a respective burr diameter, wherein a burr-to-coil diameter ratio defined by each respective burr diameter and the outer coil diameter is about 1.3-1.7.

28. The method of claim 27, wherein a distal concentric cylindrical abrasive element is fixedly mounted to an exterior of the torque-transmitting coil, the distal concentric cylindrical abrasive element having a center of mass aligned with the drive shaft axis.

29. The method of claim 28, wherein the distal concentric cylindrical abrasive element is fixed to the torque-transmitting coil and is distally spaced apart from a distal-most abrasive burr of the three or more abrasive burrs, and having an abrasive coating on an exterior of the distal concentric cylindrical abrasive element.
